(19) United States
(12) Patent Application Publication
(10) Pub. No.: US 2026/0223847 A1
(43) Pub. Date: Aug. 6, 2026

(54) Compounds for Increasing Frost Tolerance in Plants

(71) Applicants: VIB VZW, Gent (BE); Universiteit Gent, Gent (BE)

(72) Inventors: Jenny Russinova, Gent (BE); Qian Ma, Gent (BE); Johan Winne, Gent (BE)

(21) Appl. No.: 19/148,852

(22) PCT Filed: Jan. 18, 2024
(86) PCT No.: PCT/EP2024/051129

§ 371 Date: Jul. 17, 2025

(30) Foreign Application Priority Data
Jan. 18, 2023 (EP) 23152240.0

(51) Int. Cl. A01N 43/38; A01N 39/00; A01N 43/66; A01P 21/00; C07D 209/48; C07D 403/12; C07D 405/06; C07D 409/12

(52) U.S. Cl. CPC A01N 43/38; A01N 39/00; A01N 43/66; A01P 21/00; C07D 209/48; C07D 403/12; C07D 405/06; C07D 409/12

(57) ABSTRACT

The present invention provides novel agrochemicals and formulations thereof and the use of these agrochemical formulations for providing abiotic stress tolerance, such as cold and frost tolerance, in plants.

### CROSS REFERENCE TO RELATED APPLICATIONS

This application is a national phase entry under 35 U.S.C. § 371 of International Patent Application PCT/EP2024/051129, filed Jan. 18, 2024, designating the United States of America and published in English as International Patent Publication WO 2024/153736 on Jul. 25, 2024, which claims the benefit under Article 8 of the Patent Cooperation Treaty to European Patent Application Serial No. 23152240.0, filed Jan. 18, 2023, the entireties of which are hereby incorporated by reference.

### FIELD OF THE INVENTION

The present invention relates to the field of agriculture, more particularly to the field of agrochemicals. More particularly the invention provides novel agrochemicals and formulations thereof and the use of these agrochemical formulations for providing abiotic stress tolerance, such as cold and frost tolerance, in plants.

### INTRODUCTION

Human population is increasing at an alarming pace and believed to exceed 9.7 billion by 2050, whereas at the same time the agricultural productivity is decreasing due to the growing environmental constraints as a result of global climate change. Cold stress is one of the widespread abiotic stresses affecting crop productivity particularly in temperate regions. The phenotypic manifestations of cold injury in plants are highly variable. Both low temperature and rapid fluctuations between heat and cold can severely affect the physiology of plants. Cold stress inflicts damages to fruit trees, horticultural and landscape plants, as well as crop plants, posing a major threat to sustainable agriculture. Plants have developed various anatomical, physiological and genetic strategies to cope with the cold stress. Conventional breeding methods have resulted in inadequate success in improving the cold tolerance of vital crop plants through inter-specific or inter-generic hybridization. Therefore, it is of the essence to speed up the efforts for unravelling the biochemical, physiological and molecular mechanisms underlying cold stress tolerance in plants. While quite a few programs have been taken up in leading global research institutes but the pace of development of cold stress tolerant cultivars is not up to the mark when compared to ever-increasing pressure of abiotic stresses including cold stress due to global climate change. Moreover, the intricate genetic mechanisms involved in plant adaptation to cold stresses have been a key obstacle for crop improvement using conventional plant breeding tools. Omics technologies including genomics, transcriptomics and proteomics can facilitate the elucidation of complex mechanisms involved in plant adaptation to cold stress but this takes huge efforts and time. In the present invention we have identified a group of compounds which can provide cold tolerance to plants.

### FIGURE LEGENDS

FIG. 1: Left: hypocotyl length of Arabidopsis Col-0 seedlings germinated on solid ½MS medium for 4 days and then transferred for 3 more days to liquid ½MS supplemented with a range of concentrations of A68. Right: primary root length of Arabidopsis Col-0 seedlings grown on solid ½MS medium supplemented with a range of concentrations of A68 for 12 days.

FIG. 2: Effects of A68 and its related variants on the Arabidopsis hypocotyl elongation. Arabidopsis seedlings were grown on solid ½MS medium for 4 days and then transferred for 3 more days to liquid ½ MS supplemented with the studied compounds at either 0.5 or 50 μM final concentration. Hypocotyl length is represented relative to that of mock-treated seedlings from the same biological repeats. n>400 seedlings for DMSO and A68 initial hit (control treatments throughout the experiments), n>30 seedlings for the A68 variants. Student's two-sample t-test, *P<0.05, **P<0.01 and ***P<0.001.

FIG. 3: Hypocotyl elongation dose-response curves for A68 and three bioactive variants A68-6, A68-8 and A68-53 showing the half-maximum effective concentration EC₅₀.

FIGS. 4A-4D: Effect of A68 and its variant A68-6 on Arabidopsis growth in osmotic stress conditions. The two compounds were tested at 3 different concentrations in ½MS without sucrose supplemented with 100 mM sorbitol. The extent of stress tolerance in presence of the small molecules was estimated by following the rosette size (FIG. 4A) as well as root related parameters, such as the total (FIG. 4B) and main (FIG. 4C) root length and lateral root number (FIG. 4D). Results are presented relative to the mock-treated control (0.005% DMSO).

FIGS. 5A-5D: Effect of A68-6 on Arabidopsis growth in osmotic stress conditions. The compound was tested at 3 different concentrations in ½MS without sucrose supplemented with 200 mM sorbitol. The extent of stress tolerance in presence of the small molecules was estimated by following the rosette size (FIG. 5A) as well as root related parameters, such as the total (FIG. 5B) and main (FIG. 5C) root length and lateral root number (FIG. 5D). Results are presented relative to the mock-treated control (0.005% DMSO).

FIG. 6: percentage of healthy flowers on the marked branch (flowering during frost) one week after frost occurred

FIG. 7: harvest data showing the average fruit weight and the % of class 1 fruits

FIG. 8: assessment of the ripening of the berries over 3 different harvest moments

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions as described herein are provided solely to aid in the understanding of the invention. Unless specifically defined herein, all terms used herein have the same meaning as they would to one skilled in the art of the present invention. The definitions provided herein should not be construed to have a scope less than understood by a person of ordinary skill in the art.

In one embodiment the invention provides a compound depicted in formula (I)

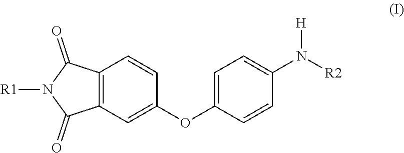

wherein R1 is H or

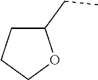

and wherein R2 is selected from the list consisting of

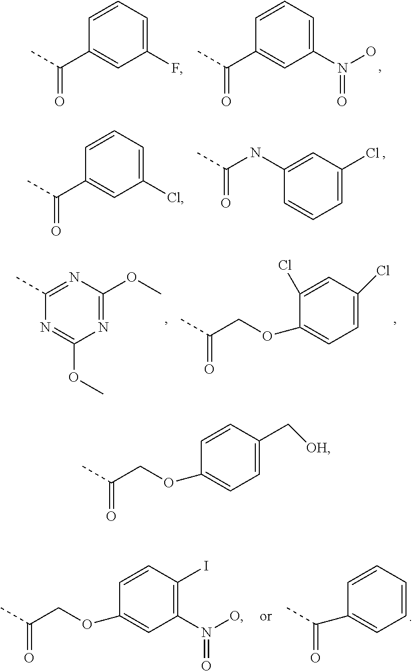

wherein the bound depicted as [symbol] connects the structure with the nitrogen (N—H group), and agronomically acceptable salts thereof, and individual enantiomers and diastereomers thereof.

In yet another embodiment the invention provides the use of a structure depicted in formula (II) as

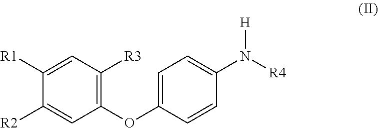

wherein R1 is H or

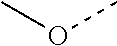

and R2 is H,
or wherein R1 and R2 together form an N-substituted maleimide structure:

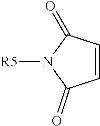

wherein R5 is H or

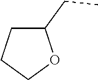

wherein the bound depicted as [symbol] connects the structure with the nitrogen (N—)
and wherein R3 is H or

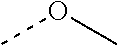

wherein the bound depicted as [symbol] connects the structure with the aryl moiety
and wherein R4 is

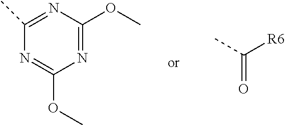

and wherein R6 is selected from the list consisting of

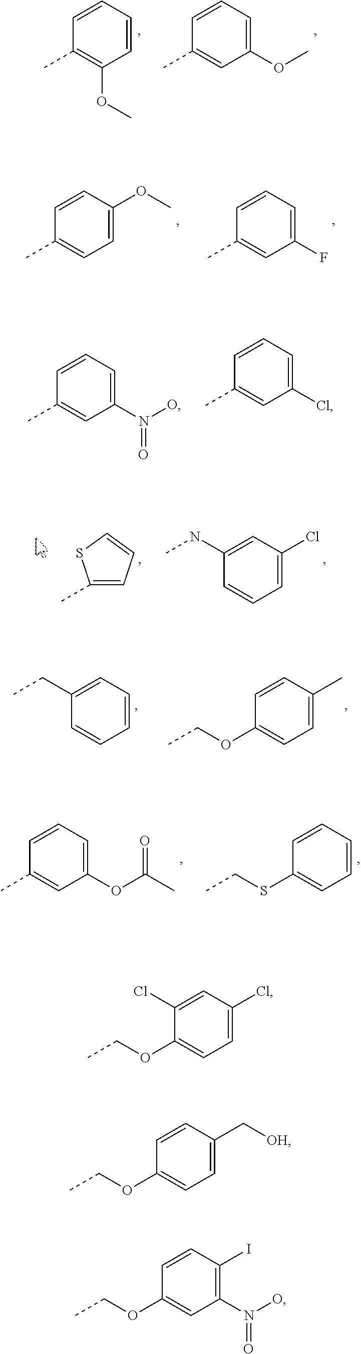

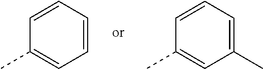

wherein the bound depicted as [symbol] connects the structures with the amide moiety, and the use of agronomically acceptable salts of these structures, and individual enantiomers and diastereomers thereof, for increasing the abiotic stress tolerance in plants.

In yet another embodiment the invention provides a compound selected from the list N-(4-((1,3-dioxoisoindolin-5-yl)oxy)phenyl)-3-fluorobenzamide, N-(4-((1,3-dioxoisoindolin-5-yl)oxy)phenyl)-3-nitrobenzamide, 3-chloro-N-(4-((1,3-dioxoisoindolin-5-yl)oxy)phenyl)benzamide, 1-(3-chlorophenyl)-3-(4-((1,3-dioxoisoindolin-5-yl)oxy)phenyl) urea, 5-(4-((4,6-dimethoxy-1,3,5-triazin-2-yl)amino) phenoxy) isoindoline-1,3-dione, 2-(2,4-dichlorophenoxy)-N-(4-((1,3-dioxoisoindolin-5-yl)oxy)phenyl)acetamide, N-(4-((1,3-dioxoisoindolin-5-yl)oxy)phenyl)-2-(4-N-(4-((1,3-dioxoisoindolin-5-yl)oxy)phenyl)-2-(3-iodo-4-(hydroxymethyl) phenoxy)acetamide, nitrophenoxy)acetamide, N-(4-((1,3-dioxoisoindolin-5-yl)oxy)phenyl)benzamide and N-(4-((1,3-dioxo-2-((tetrahydrofuran-2-yl)methyl) isoindolin-5-yl)oxy)phenyl)benzamide.

In yet another embodiment the invention provides the use of a compound selected from the list N-(4-((1,3-dioxoisoindolin-5-yl)oxy)phenyl)-3-fluorobenzamide, N-(4-((1,3-dioxoisoindolin-5-yl)oxy)phenyl)-3-nitrobenzamide, 3-chloro-N-(4-((1,3-dioxoisoindolin-5-yl)oxy)phenyl)benzamide, 1-(3-chlorophenyl)-5-(4-((4,6-dimethoxy-1,3,5-triazin-2-3-(4-((1,3-dioxoisoindolin-5-yl)oxy)phenyl) urea, yl)amino) phenoxy) isoindoline-1,3-dione, 2-(2,4-dichlorophenoxy)-N-(4-((1,3-dioxoisoindolin-5-yl)oxy)phenyl)acetamide, N-(4-((1,3-dioxoisoindolin-5-yl)oxy)phenyl)-2-(4-(hydroxymethyl) phenoxy)acetamide, N-(4-((1,3-dioxoisoindolin-5-yl)oxy)phenyl)-2-(3-iodo-4-nitrophenoxy)acetamide, N-(4-((1,3-dioxoisoindolin-5-yl)oxy)phenyl)benzamide, N-(4-((1,3-dioxo-2-((tetrahydrofuran-2-yl)methyl) isoindolin-5-yl)oxy)phenyl)benzamide, N-(4-((1,3-dioxoisoindolin-5-yl)oxy)phenyl)-3-methoxybenzamide, N-(4-((1,3-dioxoisoindolin-5-yl)oxy)phenyl)-2-methoxybenzamide, 3-methoxy-N-(4-phenoxyphenyl)benzamide, 3-methoxy-N-(4-(4-methoxyphenoxy)phenyl)benzamide, 3-methoxy-N-(4-(2-methoxyphenoxy)phenyl)benzamide, N-(4-((1,3-dioxoisoindolin-5-yl)oxy)phenyl)thiophene-2-carboxamide, N-(4-((1,3-dioxoisoindolin-5-yl)oxy)phenyl)-2-phenylacetamide, N-(4-((1,3-dioxoisoindolin-5-yl)oxy)phenyl)-2-(p-tolyloxy)acetamide, 3-((4-((1,3-dioxoisoindolin-5-yl)oxy)phenyl) carbamoyl)phenyl acetate, N-(4-((1,3-dioxo-2-((tetrahydrofuran-2-yl)methyl) isoindolin-5-yl)oxy)phenyl)-3-methoxybenzamide, N-(4-((1,3-dioxoisoindolin-5-yl)oxy)phenyl)-2-(phenylthio)acetamide, and N-(4-((1,3-dioxoisoindolin-5-yl)oxy)phenyl)-3-methylbenzamide for increasing abiotic stress tolerance in plants.

In a particular embodiment the abiotic stress tolerance is cold tolerance.

In another particular embodiment the abiotic stress tolerance is frost tolerance.

When an asymmetric center is present in a compound of formula (I) or formula (II) hereinafter referred to as a “compound of the invention,” the compound may exist in the form of optical isomers (enantiomers). In one embodiment, the present invention comprises enantiomers and mixtures, including racemic mixtures of the compounds of formula (I) and formula (II). In another embodiment, for compounds of formula (I) or formula (II) that contain more than one asymmetric center, the present invention comprises diastereomeric forms (individual diastereomers and mixtures thereof) of compounds.

The present invention comprises the tautomeric forms of compounds of formula (I) or formula (II). Where structural isomers are interconvertible via a low energy barrier, tautomeric isomerism (‘tautomerism’) can occur. This can take the form of proton tautomerism in compounds of formula (I) or formula (II) containing, for example, an imino, keto, or oxime group, or so-called valence tautomerism in compounds which contain an aromatic moiety. It follows that a single compound may exhibit more than one type of isomerism. The various ratios of the tautomers in solid and liquid form are dependent on the various substituents on the molecule as well as the particular crystallization technique used to isolate a compound.

Suitable agronomically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Furthermore, where the compounds of the invention carry an acidic moiety, suitable agronomically acceptable salts thereof may include alkali metal salts, e.g. sodium or potassium salts; alkaline earth metal salts, e.g. calcium or magnesium salts; and salts formed with suitable organic ligands, e.g. quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts. In one embodiment, hemisalts of acids and bases may also be formed, for example, hemisulphate and hemicalcium salts.

The present invention also includes isotopically labelled compounds, which are identical to those recited in formula (I) or formula (II), but for the fact that one or more atoms are replaced by an atom having an atomic mass or mass number different from the atomic mass or mass number usually found in nature. Examples of isotopes that may be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention and agronomically acceptable salts of said compounds or which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in plant tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain agronomic advantages resulting from greater stability, for example increased in half-life in the soil or plant or reduced dosage requirements and, hence, may be preferred in some circumstances, isotopically labelled compounds of formula (I) or (II) of this invention may generally be prepared by carrying out the procedures disclosed in the Schemes and/or in the Materials and Methods section below, by substituting a readily available isotopically labelled reagent for a non-isotopically labelled reagent.

As stated above, the agrochemical formulations comprising the compounds of formula (I) or (II) are used in “effective amounts”. This means that they are used in a quantity which allows to obtain the desired effect which is a (synergistic) increase of the health of a plant but which does not give rise to any phytotoxic symptom on the treated plant.

For use according to the present invention, the agrochemical formulations comprising the compounds of formula (I) or (II) can be converted into the customary formulations, for example solutions, emulsions, suspensions, dusts, powders, pastes and granules. The use form depends on the particular intended purpose; in each case, it should ensure a fine and even distribution of the agrochemical formulations comprising the compounds of formula (I) or (II) according to the present invention. The formulations are prepared in a known manner to the person skilled in the art.

The agrochemical formulations may also comprise auxiliaries which are customary in agrochemical formulations. The auxiliaries used depend on the particular application form and active substance, respectively. Examples for suitable auxiliaries are solvents, solid carriers, dispersants or emulsifiers (such as further solubilizers, protective colloids, surfactants and adhesion agents), organic and inorganic thickeners, bactericides, antifreezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g. ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wetters, tackifiers, dispersants or emulsifiers) are alkali metal, alkaline earth metal and ammonium salts of aromatic sulfonic acids, such as ligninsulfonic acid, phenolsulfonic acid, naphthalenesulfonic acid, dibutylnaphthalenesulfonic acid and fatty acids, alkylsulfonates, alkyl-arylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearyl-phenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquid and proteins, denatured proteins, polysaccharides (e.g. methylcellulose), hydrophobically modified starches, polyvinyl alcohols, polycarboxylates types, polyalkoxylates, polyvinylamines, polyvinylpyrrolidone and the copolymers thereof. Examples for thickeners (i.e. compounds that impart a modified flowability to formulations, i.e. high viscosity under static conditions and low viscosity during agitation) are polysaccharides and organic and anorganic clays such as Xan-than gum.

Bactericides may be added for preservation and stabilization of the formulation. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and ben-zisothiazolinones (Acticide® M BS from Thor Chemie). Examples for suitable antifreezing agents are ethylene glycol, propylene glycol, urea and glycerin. Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and agrochemical formulations comprising the compounds of formula (I) and (II) thereof.

Suitable colorants are pigments of low water solubility and solvent-soluble, e.g. water-soluble, dyes.

Examples for adhesion promoters, like tackifiers or binders, are polyvinylpyrrolidons, polyvinylacetates, polyvinyl alcohols and cellulose ethers (Tylose®, Shin-Etsu, Japan).

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Anticaking agents like oils and/or waxes can be added.

The agrochemical formulations generally comprise between 0.01 and 95%, preferably between 0.1 and 90%, most preferably between 0.5 and 90%, by weight of active substances. The compounds of the agrochemical formulations comprising the compounds of formula (I) or (II) are employed in a purity of from 90% to 100%, preferably from 95% to 100% (according to their NMR spectrum).

The compounds of the agrochemical formulations comprising the compounds of formula (I) or (II) can be used as such or in the form of their agricultural compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the compounds present in the agrochemical formulations comprising the compounds of formula (I) or (II).

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1%, by weight of compounds of the agrochemical formulations comprising the compounds of formula (I) or (II).

The compounds of the agrochemical formulations comprising the compounds of formula (I) or (II) may also be used successfully in the ultra-low-volume process (ULV), it being possible to apply compositions comprising over 95% by weight of active substance, or even to apply the active substance without additives.

Various types of oils, wetters, adjuvants, herbicides, fungicides, other pesticides, or bactericides may be added to the active compounds, if appropriate not until immediately prior to use (tank mix). These agents can be admixed with the compounds of the agrochemical formulations comprising the compounds of formula (I) or (II) in a weight ratio of 1:100 to 100:1, preferably 1:10 to 10:1.

Compositions of this invention may also contain fertilizers (such as ammonium nitrate, urea, potash, and superphosphate), phytotoxicants and plant growth regulators (plant growth amendments) and safeners. These may be used sequentially or in combination with the above-described compositions, if appropriate also added only immediately prior to use (tank mix). For example, the plant(s) may be sprayed with a composition of this invention either before or after being treated with the fertilizers. In a particular embodiment the agronomical compositions of the invention may be used in hydroponic conditions.

In the agrochemical formulations comprising the compounds of formula (I), the weight ratio of the compounds generally depends from the properties of the compounds of the agrochemical formulations comprising the compounds of formula (I) or (II).

The compounds of the agrochemical formulations comprising the compounds of formula (I) or (II) can be used individually or already partially or completely mixed with one another to prepare the composition according to the invention. It is also possible for them to be packaged and used further as combination composition such as a kit of parts.

The user applies the composition according to the invention usually from a pre-dosage device, a knapsack sprayer, a spray tank or a spray plane. Here, the agrochemical composition is made up with water and/or buffer to the desired application concentration, it being possible, if appropriate, to add further auxiliaries, and the ready-to-use spray liquid or the agrochemical composition according to the invention is thus obtained. Usually, 50 to 500 liters of the ready-to-use spray liquid are applied per hectare of agricultural useful area, preferably 50 to 400 liters. In a particular embodiment the composition according to the invention is added in a hydroponic culture.

In a particular embodiment the absolute amount of the active compounds, represented by formula (I) or (II), is used in a range between 1 mg/liter to 100 mg/liter, particularly in a range between 1 mg/l to 20 mg/l, particularly in a range between 1 mg/l to 25 mg/l, particularly in a range between 2 mg/l to 200 mg/l, particularly between 2 mg/l to 100 mg/l, particularly between 2 mg/l to 50 mg/l, particularly between 2 mg/l to 25 mg/l, particularly between 4 mg/l to 40 mg/l, particularly between 4 mg/l to 20 mg/l, particularly between 4 mg/l to 16 mg/l, particularly between 4 mg/l to 12 mg/l.

According to one embodiment, individual compounds of the agrochemical formulations comprising the compounds of formula (I) or (II) formulated as composition (or formulation) such as parts of a kit or parts of the inventive mixture may be mixed by the user himself in a spray tank and further auxiliaries may be added, if appropriate (tank mix).

“Agrochemical”, as used herein, means any active substance that may be used in the agrochemical industry (including agriculture, horticulture, floriculture and home and garden uses.

An “agrochemical composition” as used herein means a composition for agrochemical use, as herein defined, comprising at least one active substance of a compound of formula (I) or (II), optionally with one or more additives favoring optimal dispersion, atomization, deposition, leaf wetting, distribution, retention and/or uptake of agrochemicals. As a non-limiting example such additives are diluents, solvents, adjuvants, surfactants, wetting agents, spreading agents, oils, stickers, viscosity-adjusting agents (like thickeners, penetrants), pH-adjusting agents (like buffering agents, acidifiers), anti-settling agents, anti-freeze agents, photo-protectors, defoaming agents, biocides and/or drift control agents.

A “carrier”, as used herein, means any solid, semi-solid or liquid carrier in or on (to) which an active substance can be suitably incorporated, included, immobilized, adsorbed, absorbed, bound, encapsulated, embedded, attached, or comprised. Nonlimiting examples of such carriers include nanocapsules, microcapsules, nanospheres, microspheres, nanoparticles, microparticles, liposomes, vesicles, beads, a gel, weak ionic resin particles, liposomes, cochleate delivery vehicles, small granules, granulates, nano-tubes, bucky-balls, water droplets that are part of an water-in-oil emulsion, oil droplets that are part of an oil-in-water emulsion, organic materials such as cork, wood or other plant-derived materials (e.g. in the form of seed shells, wood chips, pulp, spheres, beads, sheets or any other suitable form), paper or cardboard, inorganic mate-rials such as talc, clay, microcrystalline cellulose, silica, alumina, silicates and zeolites, or even microbial cells (such as yeast cells) or suitable fractions or fragments thereof.

The terms “effective amount”, “effective dose” and “effective amount”, as used herein, mean the amount needed to achieve the desired result or results. More exemplary information about amounts, ways of application and suitable ratios to be used is given below. The skilled artisan is well aware of the fact that such an amount can vary in a broad range and is dependent on various factors such as the treated cultivated plant as well as the climatic and soil conditions.

As used herein, the terms “determining”, “measuring”, “assessing”, “monitoring” and “assaying” are used interchangeably and include both quantitative and qualitative determinations.

It is understood that the agrochemical composition is stable, both during storage and during utilization, meaning that the integrity of the agrochemical composition is maintained under storage and/or utilization conditions of the agrochemical composition, which may include elevated temperatures, freeze-thaw cycles, changes in pH or in ionic strength, UV-irradiation, presence of harmful chemicals and the like. More preferably, the compounds of formula (I) or (II) as herein described remain stable in the agrochemical composition, meaning that the integrity and the activity of the compounds are maintained under storage and/or utilization conditions of the agrochemical composition, which may include elevated temperatures, freeze-thaw cycles, changes in pH or in ionic strength, UV-irradiation, presence of harmful chemicals and the like. Most preferably, said compounds of formula (I) or (II) remain stable in the agrochemical composition when the agrochemical composition is stored at ambient temperature for a period of two years or when the agrochemical composition is stored at 54° C. for a period of two weeks. Preferably, the agrochemical composition of the present invention retains at least about 70% activity, more preferably at least about 70% to 80% activity, most preferably about 80% to 90% activity or more. Examples of suitable carriers include, but are not limited to alginates, gums, starch, p-cyclodextrins, celluloses, polyurea, polyurethane, polyester, or clay.

The agrochemical composition may occur in any type of formulation, preferred formulations are powders, wettable powders, wettable granules, water dispersible granules, emulsions, emulsifiable concentrates, dusts, suspensions, suspension concentrates, capsule suspensions, aqueous dispersions, oil dispersions, aerosols, pastes, foams, slurries or flowable concentrates.

In yet another embodiment the invention provides the use of the agrochemical compositions of the invention for enhancing abiotic stress tolerance in plants.

In specific embodiments the abiotic stress tolerance is cold stress in plants or frost stress in plants. Thus the compounds of the invention can be used to obtain cold stress tolerance or frost stress tolerance.

The agrochemical composition according to the invention can be applied once to a crop, or it can be applied two or more times after each other with an interval between every two applications. The agrochemical composition according to the invention can be applied alone or in mixture with other materials, preferably other agrochemical compositions, to the crop; alternatively, the agrochemical composition according to the invention can be applied separately to the crop with other materials, preferably other agrochemical compositions, applied at different times to the same crop.

In yet another embodiment the invention provides a method for the manufacture of (‘or the production of which is equivalent wording) an agrochemical composition according to the invention, comprising formulating a molecule of formula (I) or (II) as defined herein before, together with at least one customary agrochemical auxiliary agent. Suitable manufacturing methods are known in the art and include, but are not limited to, high or low shear mixing, wet or dry milling, drip-casting, encapsulating, emulsifying, coating, encrusting, pilling, extrusion granulation, fluid bed granulation, co-extrusion, spray drying, spray chilling, atomization, addition or condensation polymerization, interfacial polymerization, in situ polymerization, coacervation, spray encapsulation, cooling melted dispersions, solvent evaporation, phase separation, solvent extraction, sol-gel polymerization, fluid bed coating, pan coating, melting, passive or active absorption or adsorption.

Customary agrochemical auxiliary agents are well-known in the art and preferably include, but are not limited to aqueous and/or organic solvents, pH-adjusting agents (like buffering agents, acidifiers), surfactants, wetting agents, spreading agents, adhesion promoters (like tackifiers, stickers), carriers, fillers, viscosity-adjusting agents (like thickeners), emulsifiers, dispersants, sequestering agents, anti-settling agents, coalescing agents, rheology modifiers, defoaming agents, photo-protectors, anti-freeze agents, biostimulants (including bacterial and/or fungal inoculants or microorganisms), biocides (preferably selected from herbicides, bactericides, phytotoxicants, fungicides, pesticides and mixtures thereof), plant growth regulators, safeners, penetrants, anticaking agents, mineral and/or vegetable oils and/or waxes, colorants and drift control agents or any suitable combination thereof. Preferably, the customary agrochemical auxiliary agents are not aqueous or organic solvents.

The compounds of general formula (I) or (II) employed according to the present invention can be employed in combination with these auxiliaries. The auxiliaries used depend on the particular application form and the active substance and preferably include solvents, solid carriers, dispersants or emulsifiers, such as solubilizers, protective colloids, surfactants and adhesion agents. Furthermore, organic and inorganic thickeners, bactericides, anti-freezing agents, anti-foaming agents, if appropriate, colorants and tackifiers or binders can be employed in combination with the nitrification inhibitors and in the fertilizer mixture. Suitable auxiliaries are discussed in WO 2013/121384 on pages 25 to 26.

Further possible preferred ingredients are oils, wetters, adjuvants, biostimulants, herbicides, bactericides, other fungicides and/or pesticides. They are for example discussed in WO 2013/121384 on pages 28/29.

According to one embodiment of the invention, the auxiliaries are not solvents.

The plants to be treated or rooted in soil or the plants grown in hydroponics conditions to be treated according to the invention are preferably selected from the group consisting of agricultural, silvicultural, ornamental and horticultural plants, each in its natural or genetically modified form. Preferred agricultural plants are field crops selected from the group consisting of potatoes, sugar beets, wheat, barley, strawberries, orchids, azalea, rye, oat, sorghum, rice, maize, cotton, rapeseed, oilseed rape, canola, soybeans, peas, field beans, sunflowers, sugar cane; cucumbers, tomatoes, onions, leeks, lettuce, squashes; even more preferably the plant is selected from the group consisting of wheat, barley, oat, rye, soybean, maize, oilseed rape, cotton, sugar cane, rice and sorghum.

In a preferred embodiment of the invention, the plant to be treated is selected from the group consisting of tomato, potato, wheat, barley, oat, rye, soybean, maize, oilseed rape, canola, sunflower, cotton, sugar cane, sugar beet, rice, sorghum, pasture grass and grazed land.

In another preferred embodiment of the invention, the plant to be treated is selected from the group consisting of tomato, potato, wheat, barley, oat, rye, soybean, maize, oilseed rape, canola, sunflower, cotton, sugar cane, sugar beet, rice and sorghum.

In an especially preferred embodiment of the invention, the plants to be treated are selected from the group consisting of tomato, wheat, barley, oat, rye, maize, oilseed rape, canola, sugar cane, and rice.

In one embodiment, the plant to be treated according to the method of the invention is an agricultural plant. “Agricultural plants” are plants of which a part (e.g. seeds) or all is harvested or cultivated on a commercial scale or which serve as an important source of feed, food, fibres (e.g. cotton, linen), combustibles (e.g. wood, bioethanol, biodiesel, biomass) or other chemical compounds. Preferred agricultural plants are for example cereals, e.g. wheat, rye, barley, triticale, oats, sorghum or rice, beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rapeseed, oilseed rape, canola, linseed, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as maize, soybean, rapeseed, canola, sugar cane or oil palm; tobacco; nuts; coffee; tea; bananas; vines (table grapes and grape juice grape vines); hop; turf; natural rubber plants.

Pasture grass and grassland are composed of grass or grass mixtures comprising for example Bluegrass (Poa spp.), Bentgrass (Agrostis spp.), Ryegrasses (Lolium spp.), Fescues (Festuca spp., hybrids, and cultivars), Zoysiagrass (Zoysia spp.), Bermu-dagrass (Cynodon spp.), St. Augustine grass, Bahiagrass (Paspalum), Centipedegrass (Eremachloa), Carpetgrass (Axonopus), Buffalograss and Grama grass. Pastures may be also composed of mixtures comprising afore mentioned grasses, for example Ryegrass, and Trifolium species, for example Trifolium pratensis and Trifolium repens, Medicago species like Medicago sativa, Lotus species like Lotus corniculatus, and Melilotus species, for example Melilotus albus.

In one embodiment, the plant to be treated according to the method of the invention is a horticultural plant. The term “horticultural plants” are to be understood as plants which are commonly used in horticulture—e.g. the cultivation of ornamentals, herbs, vegetables and/or fruits. Examples for ornamentals are turf, geranium, azalea, orchids, pelargonia, petunia, begonia and fuchsia. Examples for vegetables are potatoes, tomatoes, peppers, cucurbits, cucumbers, melons, watermelons, garlic, onions, carrots, cabbage, beans, peas and lettuce and more preferably from tomatoes, onions, peas and lettuce. Examples for fruits are apples, pears, cherries, strawberry, citrus, peaches, apricots and blueberries. In horticulture, often a substrate replaces (part of) the soil.

In one embodiment, the plant to be treated according to the method of the invention is an ornamental plant. “Ornamental plants” are plants which are commonly used in gardening, e.g. in parks, gardens and on balconies. Examples are turf, geranium, pelargonia, petunia, begonia and fuchsia.

In one embodiment, the plant to be treated according to the method of the invention is a silvicultural plant. The term “silvicultural plant” is to be understood as trees, more specifically trees used in reforestation or industrial plantations. Industrial plantations generally serve for the commercial production of forest products, such as wood, pulp, paper, rubber tree, Christmas trees, or young trees for gardening purposes. Examples for silvicultural plants are conifers, like pines, in particular Pinus spec, fir and spruce, eucalyptus, tropical trees, like teak, rubber tree, oil palm, willow (Salix), in particular Salix spec, poplar (cottonwood), in particular Populus spec, beech, in particular Fagus spec, birch, oil palm, and oak.

The following definitions apply:

The term “plants” is to be understood as plants of economic importance and/or men-grown plants. They are preferably selected from agricultural, silvicultural, ornamental and horticultural plants, each in its natural or genetically modified form. The term “plant” as used herein includes all parts of a plant, such as germinating seeds, emerging seedlings, herbaceous vegetation, as well as established woody plants, including all belowground portions (such as the roots) and aboveground portions.

The term “soil” is to be understood as a natural body comprised of living (e.g. microorganisms (such as bacteria and fungi), animals and plants) and non-living matter (e.g. minerals and organic matter (e.g. organic compounds in varying degrees of decomposition), liquid, and gases) that occurs on the land surface, and is characterized by soil horizons that are distinguishable from the initial material as a result of various physical, chemical, biological, and anthropogenic processes.

### EXAMPLES

### 1. Identification of Compound A68

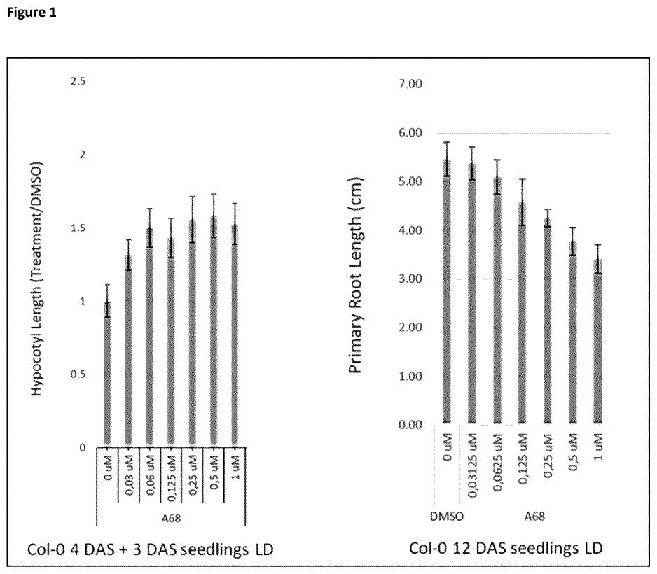

A bioactive compound, herein further designated as A68, was identified by screening a commercial 10,000 compound library (DIVERSet, ChemBridge Corporation) for hypocotyl elongation phenotype at a final concentration of 50 μM in 2% DMSO. A68 promotes hypocotyl elongation, while suppresses primary root growth but increases lateral root number under long day (LD: 16/8 h) conditions (see FIG. 1).

### 2. Structure-Activity-Relationship (SAR) Analysis of A68

A total number of sixty (60) A68 variants were used for SAR analysis based on the Arabidopsis hypocotyl growth assay outlined in example 1.

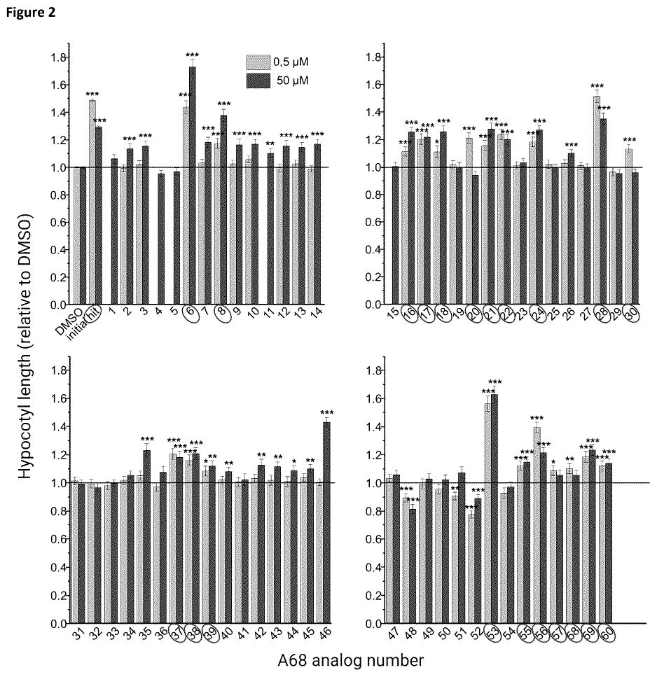

Twenty-one compounds plus A68 exhibiting significant effect at 0.5 μM were marked in red circles (see FIG. 2). Their molecular structures are shown in Table 1.

TABLE 1

| Bioactive | | |
|---|---|---|
| A68 Analogs | Vendor | Structure |
| A68-6 | ChemBridge | 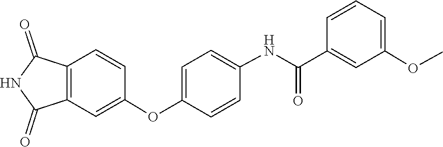 [SMILES: COc1cccc(C(=O)Nc2ccc(Oc3ccc4c(c3)C(=O)NC4=O)cc2)c1] |
| A68-8 | ChemBridge | 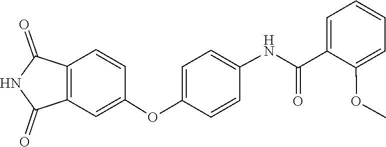 [SMILES: COc1ccccc1C(=O)Nc1ccc(Oc2ccc3c(c2)C(=O)NC3=O)cc1] |
| A68-16 | MolPort (supplied by Specs) | 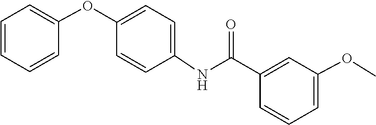 [SMILES: COc1cccc(C(=O)Nc2ccc(Oc3ccccc3)cc2)c1] |
| A68-17 | ChemBridge | 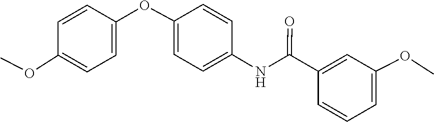 |
| A68-18 | ChemBridge | 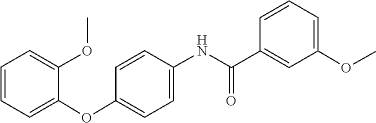 [SMILES: COc1cccc(C(=O)Nc2ccc(Oc3ccccc3OC)cc2)c1] |
| A68-20 | not applicable (N.A.) | 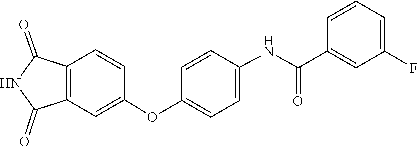 [SMILES: O=C(Nc1ccc(Oc2ccc3c(c2)C(=O)NC3=O)cc1)c1cccc(F)c1] |
| A68-21 | N.A. | 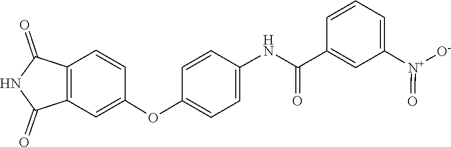 [SMILES: O=C(Nc1ccc(Oc2ccc3c(c2)C(=O)NC3=O)cc1)c1cccc([N+](=O)[O-])c1] |
| A68-22 | N.A. | 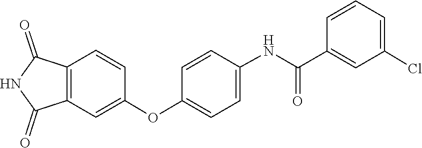 [SMILES: O=C(Nc1ccc(Oc2ccc3c(c2)C(=O)NC3=O)cc1)c1cccc(Cl)c1] |
| A68-24 | ChemBridge | 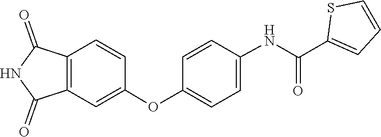 [SMILES: O=C(Nc1ccc(Oc2ccc3c(c2)C(=O)NC3=O)cc1)c1cccs1] |
| A68-28 | N.A. | 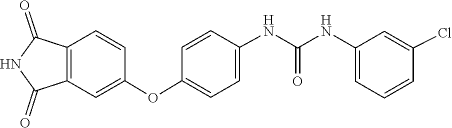 [SMILES: O=C(Nc1ccc(Oc2ccc3c(c2)C(=O)NC3=O)cc1)Nc1cccc(Cl)c1] |
| A68-30 | N.A. | 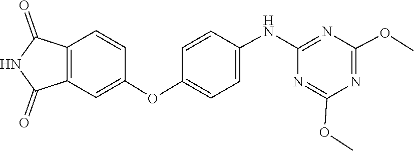 [SMILES: COc1nc(Nc2ccc(Oc3ccc4c(c3)C(=O)NC4=O)cc2)nc(OC)n1] |
| A68-37 | ChemBridge | 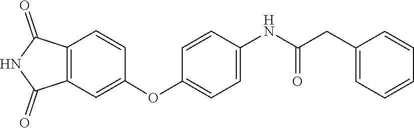 [SMILES: O=C(Cc1ccccc1)Nc1ccc(Oc2ccc3c(c2)C(=O)NC3=O)cc1] |
| A68-38 | MolPort (supplied by Vitas M) | 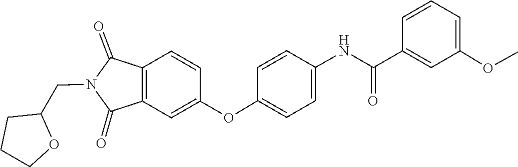 [SMILES: COc1cccc(C(=O)Nc2ccc(Oc3ccc4c(c3)C(=O)N(CC3CCCO3)C4=O)cc2)c1] |
| A68-39 | MolPort (supplied by Vitas M) | 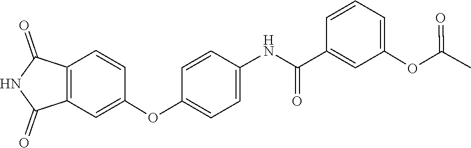 [SMILES: CC(=O)Oc1cccc(C(=O)Nc2ccc(Oc3ccc4c(c3)C(=O)NC4=O)cc2)c1] |
| A68-53 | Ambinter | 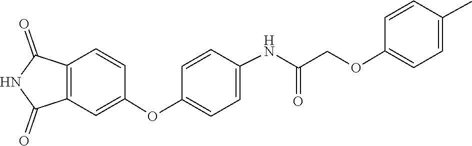 [SMILES: Cc1ccc(OCC(=O)Nc2ccc(Oc3ccc4c(c3)C(=O)NC4=O)cc2)cc1] |
| A68-55 | ChemBridge | 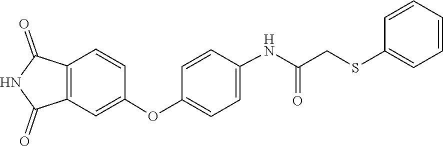 [SMILES: O=C(CSc1ccccc1)Nc1ccc(Oc2ccc3c(c2)C(=O)NC3=O)cc1] |
| A68-56 | N.A. | 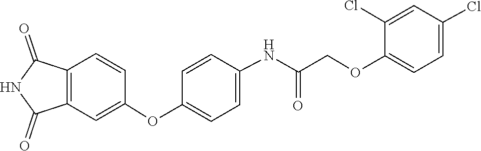 [SMILES: O=C(COc1ccc(Cl)cc1Cl)Nc1ccc(Oc2ccc3c(c2)C(=O)NC3=O)cc1] |
| A68-57 | N.A. | 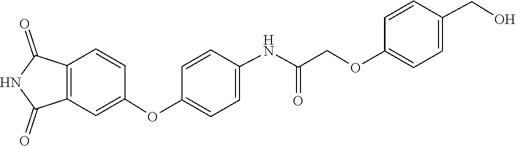 [SMILES: O=C(COc1ccc(CO)cc1)Nc1ccc(Oc2ccc3c(c2)C(=O)NC3=O)cc1] |
| A68-58 | N.A. | 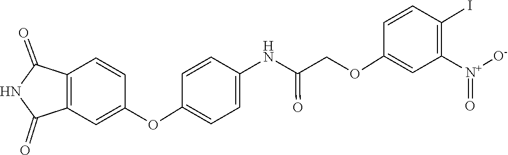 [SMILES: O=C(COc1ccc(I)c([N+](=O)[O-])c1)Nc1ccc(Oc2ccc3c(c2)C(=O)NC3=O)cc1] |
| A68-59 | N.A. | 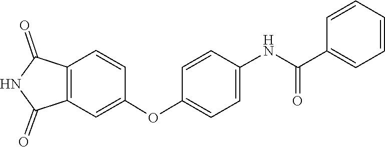 [SMILES: O=C(Nc1ccc(Oc2ccc3c(c2)C(=O)NC3=O)cc1)c1ccccc1] |
| A68-60 | N.A. | 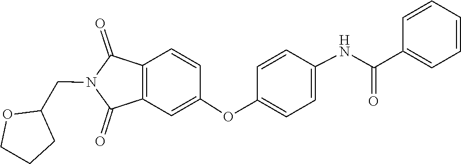 [SMILES: O=C(Nc1ccc(Oc2ccc3c(c2)C(=O)N(CC2CCCO2)C3=O)cc1)c1ccccc1] |
| A68 | ChemBridge | 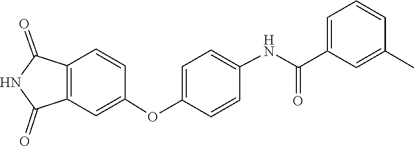 [SMILES: Cc1cccc(C(=O)Nc2ccc(Oc3ccc4c(c3)C(=O)NC4=O)cc2)c1] |

### 3. High Potency of A68 and its Variants

Hypocotyl elongation dose-response curves for A68 and three bioactive variants A68-6, A68-8 and A68-53 showing the half-maximum effective concentration EC₅₀ are depicted in FIG. 3.

### 4. A68 and Variants: Effect on Osmotic Stress in Arabidopsis

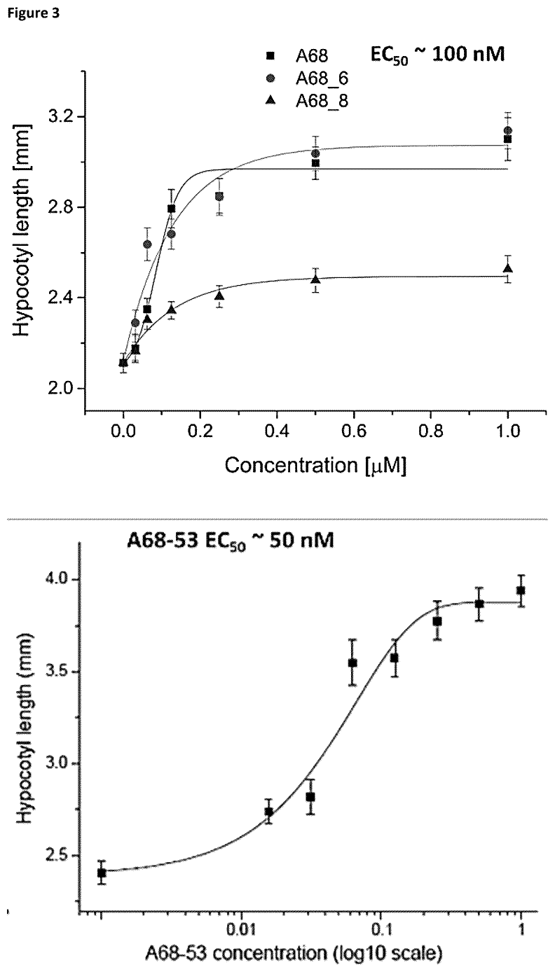

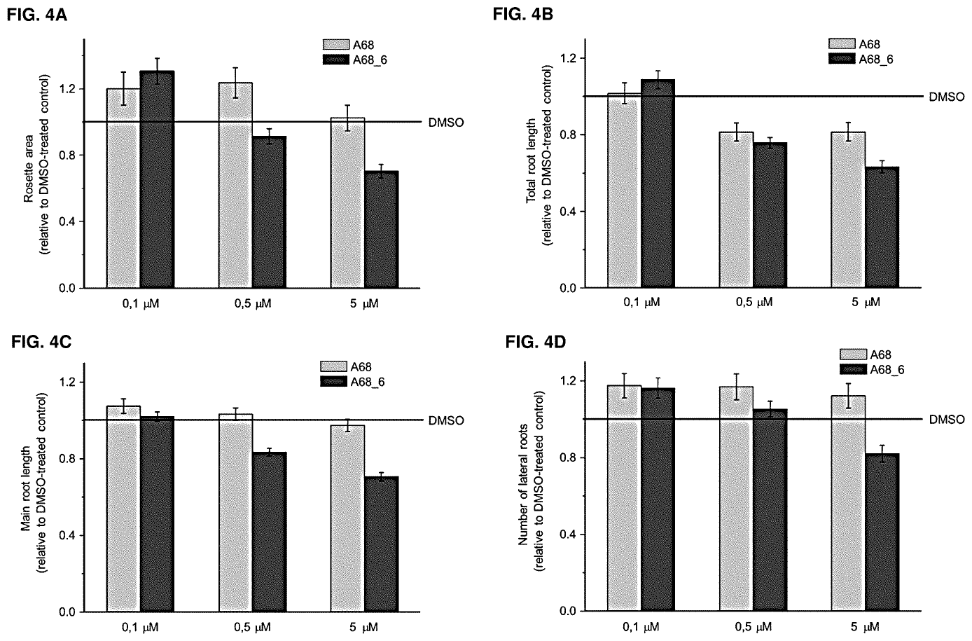

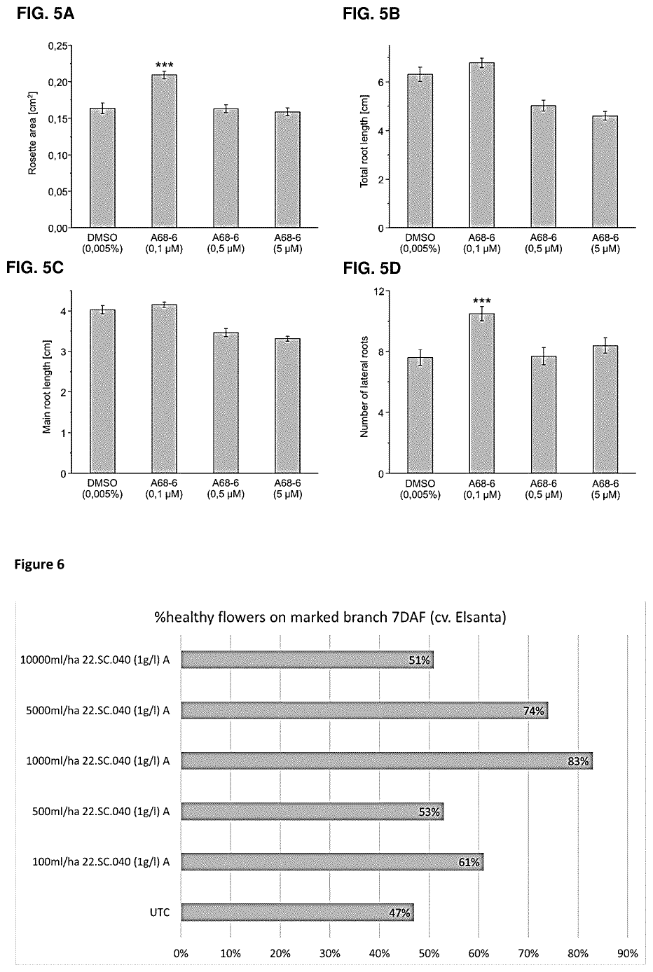

A68 and variants enhance tolerance to mild osmotic stress in Arabidopsis seedlings in low concentrations (see FIGS. 4 and 5).

### 5. Efficacy Trials of Compound A68 Against Frost Damage on Strawberry

In this example we sought to investigate whether A68 treatment enhances freezing tolerance in strawberries.

### Materials and Methods Specific for Example 5

Preparation of the agronomical formulation: compound A68 is suspended in acetone at a concentration of 1 g/l. Further in this example this suspension is designated as 22.SC.040.

Trial setup: Strawberry, Fragaria ananasa, tray plants cv. Elsanta were planted in P13 pots, 1 plant per pot. The used substrate was a standard strawberry-substrate with addition of 180 g Osmocoat/651 substrate. (Osmocoat 18-5-11+4CaO+2MgO Agroblen 3-4 months).

36 plants (6 objects×6 repetitions) were randomly placed on tables with eb-flood system (21 l/2.5 m2 during 10 minutes, 2×/24 hours) in the greenhouse (22° C./10° C. day/night; 50% RH).

Foliar applications were executed in a standardized spraying cabin. A nozzle (Albuz TVI, color green) moved over the plants with a constant speed, simulating a spraying volume of 300l/ha.

At full bloom of the first flower branch, these branches were marked with a plastic strip. The plants were placed on pallets and enter a fridge at 5° C. during night, to decrease warmth in the substrate and chill the plants. The next morning, the plants were moved from the fridge to a freezing container. When the temperature reached around-3.5° C., the plants stayed in this freezer for two hours. After this freezing period, the plants moved back to the greenhouse on the eb-flood table.

One day and one week after frost, the flowers on the marked branches were assessed. The flowers were split up in three classes during the first assessment (healthy, dead and bud stage) an only in two classes (healthy and dead) during the second assessment.

When the fruits ripen, the ripe fruits were picked. This was done weekly, till all the fruits on the marked branch were picked. Fruits were kept separately from the marked branch and fruits from the other branches. The fruits were sorted in class 1 and class 2 fruits. Class 2 fruits were malformed fruits, caused by frost damage. Also, the number of fruits were counted and the weight was measured.

The last assessments were the count of green fruits left, amount of flower branches and stolons.

The trial 22.RM.SAB.028 is a dose trial of 22.SC.040, sprayed eight days before frost. The Table 2 depicts the dose rates in this trial.

TABLE 2
dose rates used in the trial

| Code | ml/ha |
|---|---|
| 22.SC.040 (1 g/l) | 100 |
| 22.SC.040 (1 g/l) | 500 |
| 22.SC.040 (1 g/l) | 1.000 |
| 22.SC.040 (1 g/l) | 5.000 |
| 22.SC.040 (1 g/l) | 10.000 |

### Results of the RM.SAB.028 Trial

FIG. 6 shows the percentage of healthy flowers on the marked branch (flowering during frost) one week after frost occurred. A clear visible Gauss-curve appears. With a maximum percentage of healthy flowers around 1000 ml/ha or 1 g/ha 22.SC.040. This dose increases the amount of healthy flowers by 36% in this trial. Also, no negative effects in higher or lower dose-rates are assessed.

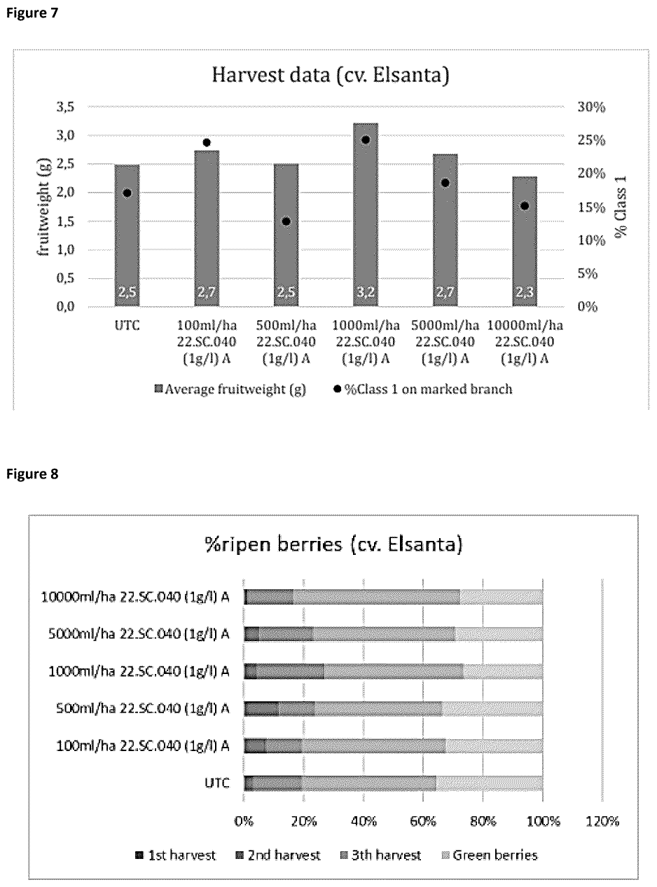

In the harvest data, an added value of 28% average fruit weight is measured at the optimal dose (1000 ml/ha) 22.SC.040. In addition, the percentage class 1 fruits is the highest at this dose range. This higher portion class 1 has an effect on fruit weight. Class 2 fruits will always have a lower fruit weight, by which the average fruit weight will decrease. FIG. 7 shows the average fruit weight and % class 1 fruits.

Also, the ripening of the berries was assessed over three different harvest moments (see FIG. 8). When the two first harvests are assessed, the Gauss-curve of the dose-range is visible with 1000 ml/ha as maximum. After the third harvest, this Gauss-curve is not visible anymore. An explanation for this is the damaged flowers by frost which would grow out to ripen berries at the first and second harvest, but now couldn't grow out (see % green berries).

We conclude that an agrochemical formulation was produced by suspending compound A68 in acetone. The trial shows clear indications of efficacy to decrease frost damage on strawberry. The dose-rates do show a positive trend, having a Gauss-curve with maximum efficacy at a certain dose-rate. These results indicate the optimal dose-rate situates around 1000 mg/ha.

### Materials and Methods

### Chemical Synthesis of the Compounds

### N-(4-((1,3-dioxoisoindolin-5-yl)oxy)phenyl)-3-fluorobenzamide (A68-20)

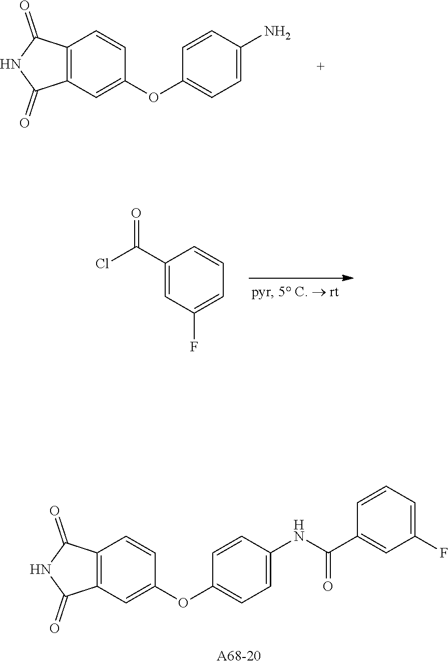 [SMILES: Nc1ccc(Oc2ccc3c(c2)C(=O)NC3=O)cc1 ; O=C(Cl)c1cccc(F)c1 ; O=C(Nc1ccc(Oc2ccc3c(c2)C(=O)NC3=O)cc1)c1cccc(F)c1]

A solution of 3-fluorobenzoyl chloride (2.6 μL, 22 μmol) in pyridine (50 μL) was added to a cooled (5° C.) solution of 5-(4-aminophenoxy) isoindoline-1,3-dione (5 mg, 20 μmol) in pyridine (50 μL). The resulting mixture was allowed to warm until room temperature. When TLC (eluent: ethyl acetate) indicated full consumption of the starting material, the mixture was concentrated in vacuo, the residue was dissolved in CHCl₃ and washed with an aqueous solution of hydrochloric acid (1M), and then with brine. The organic layer was dried over anhydrous sodium sulfate and concentrated in vacuo. The remaining solid white residue was filtered over a pad of silica gel (eluent: ethyl acetate) and this yielded A68-20 as a white solid.

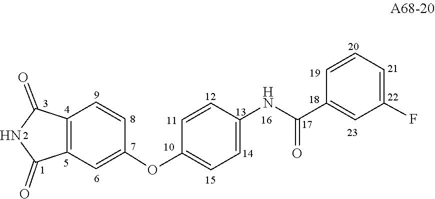

¹H NMR (aceton-d₆, 300 MHz): 10.03 (1H, br s, N16-H), 7.97 (2H, d, J=8.9 Hz, C12-H and C14-H), 7.84-7.91 (1H, band, C19-H), 7.84 (1H, d, J=8.0 Hz, 1H, C9-H), 7.43-7.80 (3H, band, C20-H, C21-H and C23-H), 7.40 (1H, dd, J=8.0, 2.3 Hz, C6-H), 7.26 (1H, d, J=2.2 Hz, C8-H), 7.22 (2H, d, J=9.0, C11-H and C15-H)

### N-(4-((1,3-dioxoisoindolin-5-yl)oxy)phenyl)-3-nitrobenzamide (A68-21)

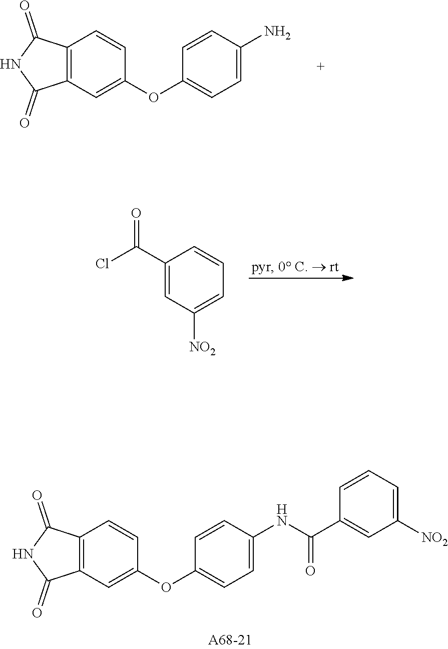 [SMILES: Nc1ccc(Oc2ccc3c(c2)C(=O)NC3=O)cc1 ; O=C(Cl)c1cccc([N+](=O)[O-])c1 ; O=C(Nc1ccc(Oc2ccc3c(c2)C(=O)NC3=O)cc1)c1cccc([N+](=O)[O-])c1]

A solution of 3-nitrobenzoyl chloride (4 mg, 22 μmol) in pyridine (50 μL) was added to a cooled (0° C.) solution of 5-(4-aminophenoxy) isoindoline-1,3-dione (5 mg, 20 μmol) in pyridine (50 μL). The resulting mixture was allowed to warm until room temperature. When TLC (eluent: ethyl acetate) indicated full consumption of the starting material, the mixture was concentrated in vacuo, the residue was dissolved in CHCl₃ and washed with an aqueous solution of hydrochloric acid (1M), and then with brine. The organic layer was dried over anhydrous sodium sulfate and concentrated in vacuo. The remaining solid white residue was filtered over a pad of silica gel (eluent: ethyl acetate) and this yielded A68-21 as a white solid.

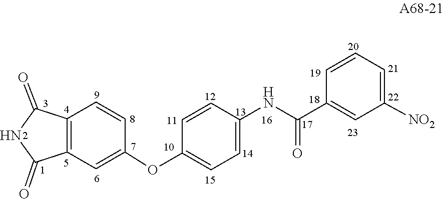

¹H NMR (aceton-d₆, 300 MHz): 10.05 (1H, br s, N16-H), 8.83 (1H, dd, J=2.6, 1.9 Hz, 1H, C23-H), 8.42-8.54 (2H, band, C19-H and C21-H), 7.97 (2H, d, J=8.9 Hz, C12-H and C14-H), 7.87 (1H, td, J=7.9, 2.4 Hz, C20-H), 7.85 (1H, d, J=8.3 Hz, C9-H), 7.41 (1H, dd, J=8.3, 2.3 Hz, C6-H), 7.27 (1H, d, J=2.3 Hz, C8-H), 7.23 (1H, d, J=8.9 Hz, C11-H and C15-H)

### 3-chloro-N-(4-((1,3-dioxoisoindolin-5-yl)oxy)phenyl)benzamide (A68-22)

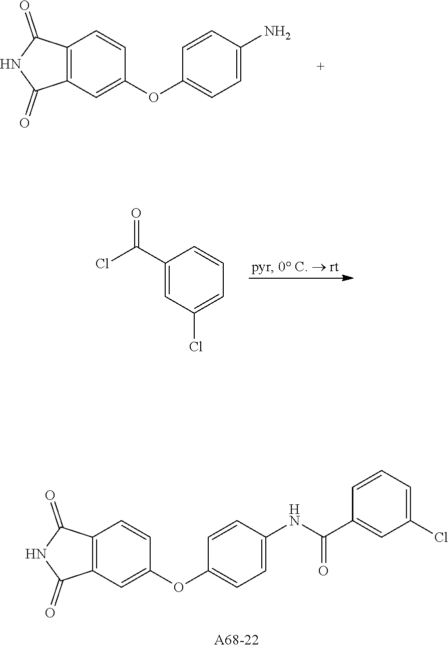 [SMILES: Nc1ccc(Oc2ccc3c(c2)C(=O)NC3=O)cc1 ; O=C(Cl)c1cccc(Cl)c1 ; O=C(Nc1ccc(Oc2ccc3c(c2)C(=O)NC3=O)cc1)c1cccc(Cl)c1]

A solution of 3-chlorobenzoyl chloride (2.8 μL, 22 μmol) in pyridine (50 μL) was added to a cooled (0° C.) solution of 5-(4-aminophenoxy) isoindoline-1,3-dione (5 mg, 20 μmol) in pyridine (50 μL). The resulting mixture was allowed to warm until room temperature. When TLC (eluent: EtOAc) indicated full consumption of the starting material, the mixture was concentrated in vacuo, the residue was dissolved in CHCl₃ and washed with an aqueous solution of hydrochloric acid (1M), and then with brine. The organic layer was dried over anhydrous sodium sulfate and concentrated in vacuo. The remaining solid white residue was filtered over a pad of silica gel (eluent: ethyl acetate) and this yielded A68-22 as a white solid.

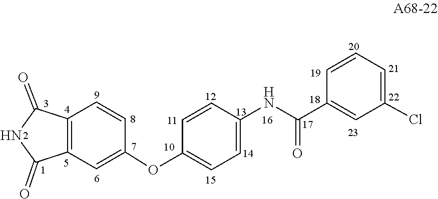

¹H NMR (aceton-d₆, 300 MHz): 10.04 (1H, br s, N16-H), 7.94-8.04 (1H, band, C19-H)), 7.97 (1H, d, J=9.0 Hz, C12-H and C14-H), 7.84 (1H, d, J=8.3 Hz, C9-H), 7.44-7.78 (3H, band, C20-H, C21-H and C23-H), 7.39 (1H, dd, J=8.3, 2.3 Hz, C6-H), 7.26 (1H, d, J=2.3 Hz, C8-H), 7.21 (1H, d, J=9.0 Hz, C11-H and C15-H).

### 1-(3-chlorophenyl)-3-(4-((1,3-dioxoisoindolin-5-yl)oxy)phenyl) urea (A68-28)

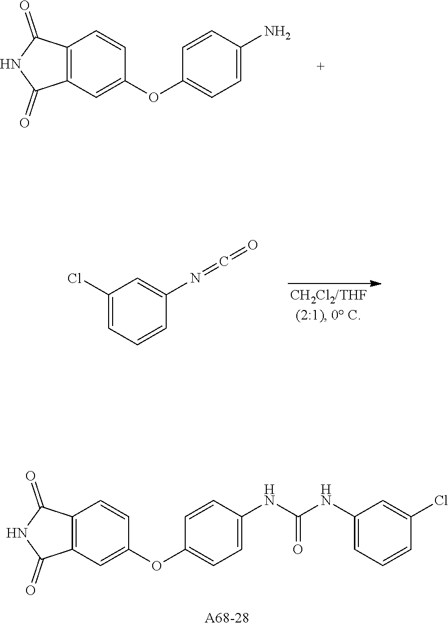 [SMILES: Nc1ccc(Oc2ccc3c(c2)C(=O)NC3=O)cc1 ; O=C=Nc1cccc(Cl)c1 ; O=C(Nc1ccc(Oc2ccc3c(c2)C(=O)NC3=O)cc1)Nc1cccc(Cl)c1]

To a solution of 5-(4-aminophenoxy) isoindoline-1,3-dione (25.4 mg, 0.1 mmol) in CH₂Cl₂ (5 mL) and THF (2.5 mL) at 0° C., 3-chlorophenyl isocyanate (18 μL, 0.15 mmol) was added. After removal of the solvent, the remaining white solid was filtered over a pad of silica (eluent: ethyl acetate) which afforded A68-28 as a white solid.

### 5-(4-((4,6-dimethoxy-1,3,5-triazin-2-yl)amino) phenoxy) isoindoline-1,3-dione (A68-30)

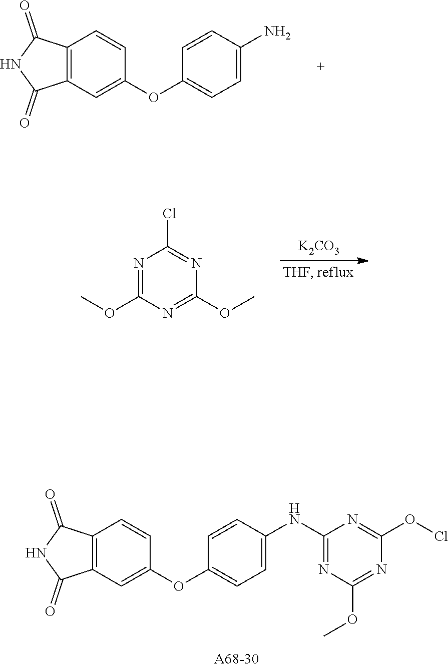

A mixture of 5-(4-aminophenoxy) isoindoline-1,3-dione (25.4 mg, 0.1 mmol), anhydrous potassium carbonate (41.5 mg, 0.3 mmol) and 2-chloro-4,6-dimethoxy-1,3,5-triazine (19.3 mg, 0.11 mmol) in THF (3 mL) was heated and stirred under reflux conditions for 30 minutes. After this the solvent was removed in vacuo, and the solid white residue was directly subjected to flash chromatography purification over a pad of silica gel (eluent: ethyl acetate), which yielded A68-30 as a white solid (5.35 mg, 39%).

### 2-(2,4-dichlorophenoxy)-N-(4-((1,3-dioxoisoindolin-5-yl)oxy)phenyl)acetamide (A68-56)

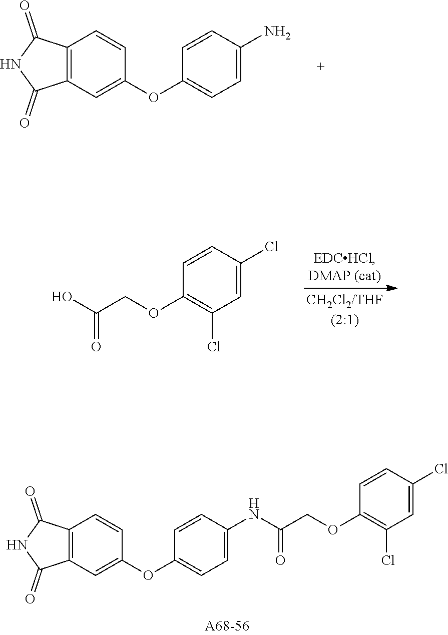 [SMILES: Nc1ccc(Oc2ccc3c(c2)C(=O)NC3=O)cc1 ; O=C(O)COc1ccc(Cl)cc1Cl ; O=C(COc1ccc(Cl)cc1Cl)Nc1ccc(Oc2ccc3c(c2)C(=O)NC3=O)cc1]

To a mixture of 5-(4-aminophenoxy) isoindoline-1,3-dione (25.4 mg, 0.1 mmol), dichlorophenoxyacetic acid (26.5 mg, 0.12 mmol), and 1-ethyl-3-(3-dimethylaminopropyl) carbodiimide hydrochloride (28.8 mg, 0.15 mmol) in dichloromethane (2 mL) and tetrahydrofuran (1 ml), a single granule of 4-dimethylaminopyridine (~2 mg) was added. The resulting mixture was stirred at room temperature for 16 h, until the reaction was finished according to TLC. The solvent was concentrated in vacuo, and the residue was purified via flash chromatography over silica gel (eluting with 20% tetrahydrofuran in dichloromethane). This yielded A68-56 (4.46 mg, 10%) as a white solid.

### N-(4-((1,3-dioxoisoindolin-5-yl)oxy)phenyl)-2-(4-(hydroxymethyl) phenoxy)acetamide (A68-57)

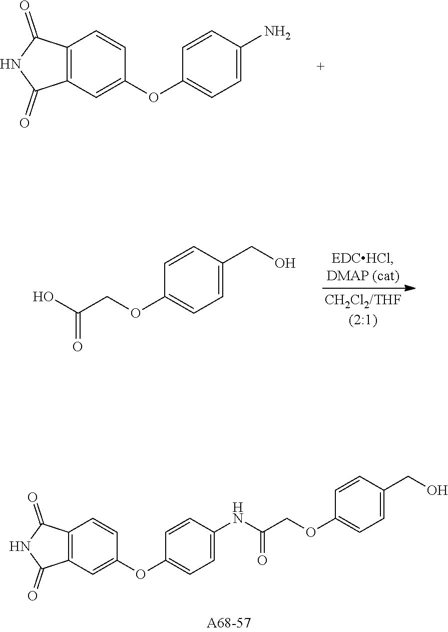 [SMILES: Nc1ccc(Oc2ccc3c(c2)C(=O)NC3=O)cc1 ; O=C(O)COc1ccc(CO)cc1 ; O=C(COc1ccc(CO)cc1)Nc1ccc(Oc2ccc3c(c2)C(=O)NC3=O)cc1]

To a mixture of 5-(4-aminophenoxy) isoindoline-1,3-dione (25.4 mg, 0.1 mmol), 4-(hydroxymethyl) phenoxyacetic acid (21.9 mg, 0.12 mmol), 1-ethyl-3-(3-dimethylaminopropyl) carbodiimide hydrochloride (28.8 mg, 0.15 mmol) in CH₂Cl₂ (2 mL) and tetrahydrofuran (1 mL), a single granule of 4-dimethylaminopyridine (~2 mg) was added. When the reaction was finished, the solvent was concentrated in vacuo. Flash chromatography purification (eluting with 10% tetrahydrofuran in dichloromethane) yielded A68-57 (2.4 mg, 6%) as a pure compound.

### N-(4-((1,3-dioxoisoindolin-5-yl)oxy)phenyl)-2-(3-iodo-4-nitrophenoxy)acetamide (A68-58)

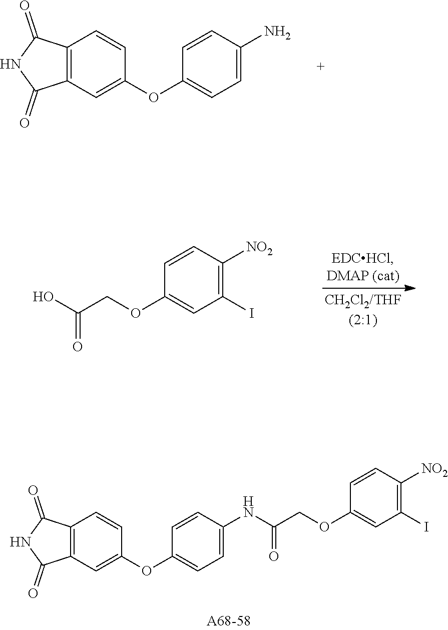 [SMILES: Nc1ccc(Oc2ccc3c(c2)C(=O)NC3=O)cc1 ; O=C(O)COc1ccc([N+](=O)[O-])c(I)c1 ; O=C(COc1ccc([N+](=O)[O-])c(I)c1)Nc1ccc(Oc2ccc3c(c2)C(=O)NC3=O)cc1]

To a mixture of 5-(4-aminophenoxy) isoindoline-1,3-dione (25.4 mg, 0.1 mmol), 4-iodo-3-nitrophenoxyacetic acid (38.8 mg, 0.12 mmol), 1-ethyl-3-(3-dimethylaminopropyl) carbodiimide hydrochloride (28.8 mg, 0.15 mmol) in dichloromethane (2 mL) and tetrahydrofuran (1 mL), one granule of 4-dimethylaminopyridine (~2 mg) was added. When the reaction was finished, the solvent was concentrated in vacuo. Flash chromatography purification (eluting with dichloromethane and a gradient of tetrahydrofuran) yielded A68-58 (3.8 mg, 7%) as a white solid.

### N-(4-((1,3-dioxoisoindolin-5-yl)oxy)phenyl)benzamide (A68-59)

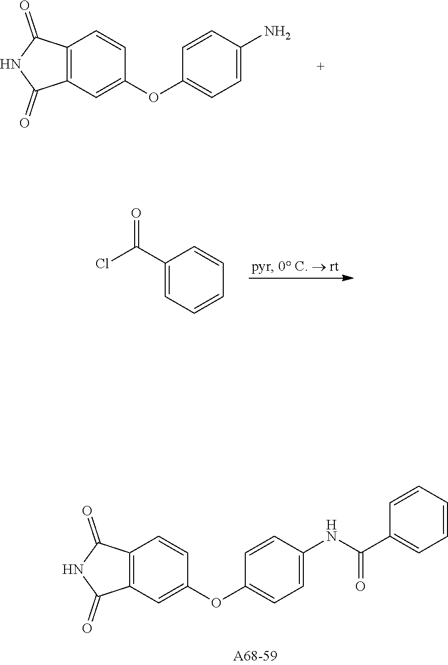 [SMILES: Nc1ccc(Oc2ccc3c(c2)C(=O)NC3=O)cc1 ; O=C(Cl)c1ccccc1 ; O=C(Nc1ccc(Oc2ccc3c(c2)C(=O)NC3=O)cc1)c1ccccc1]

A solution of benzoyl chloride (2.3 μL, 22 μmol) in pyridine (50 μL) was added to a cooled (0° C.) solution of 5-(4-aminophenoxy) isoindoline-1,3-dione (5 mg, 20 μmol) in pyridine (50 μL). The mixture was allowed to warm until room temperature. When TLC (eluent: ethyl acetate) indicated full consumption of the starting material, the mixture was concentrated in vacuo, and the residue was dissolved in chloroform. The organic layer was washed with an aqueous solution of hydrochloric acid (1M) and with brine, and then dried over anhydrous sodium sulfate and concentrated in vacuo. The solid white residue was filtered over a pad of silica gel (eluent: ethyl acetate) which afforded the title compound A68-59 as a white solid.

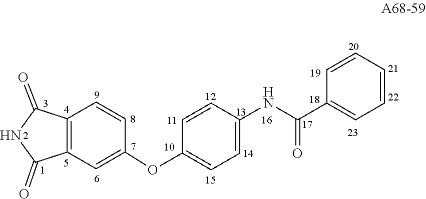

¹H NMR (aceton-d₆, 300 MHz): 10.03 (1H, br s, N16-H), 7.95-8.08 (4H, band, C12-H, C14-H, C19-H and C23-H), 7.84 (1H, d, J=8.2 Hz, C9-H), 7.47-7.67 (3H, band, C20-H, C21-H and C22-H), 7.39 (1H, dd, J=8.2, 2.3 Hz, C6-H), 7.26 (1H, d, J=2.2 Hz, C8-H), 7.21 (2H, d, J=9.0 Hz, C11-H and C15-H).

### N-(4-((1,3-dioxo-2-((tetrahydrofuran-2-yl)methyl) isoindolin-5-yl)oxy)phenyl)benzamide (A68-60)

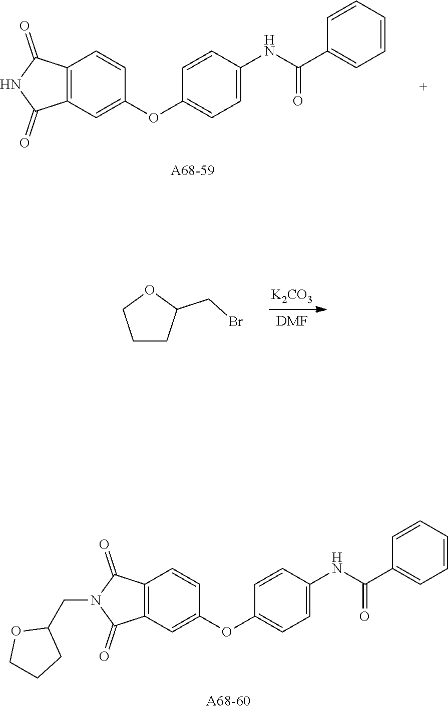

A mixture of A68-59 (48 mg, 0.134 mmol) and anhydrous potassium carbonate (37 mg, 0.268 mmol) in dimethylformamide (2 mL) was heated to 80° C. for 30 minutes under an inert atmosphere. The reaction was cooled to room temperature, and then furfuryl bromide (30 μL, 0.268 mmol) was added and the resulting mixture was again warmed to 100° C. When TLC indicated full consumption of the starting material, the solvent was removed in vacuo. Flash chromatography purification over a pad of silica gel (eluting with 4% tetrahydrofuran in dichloromethane) yielded the title compound A68-60 as a white solid (1.76 mg, 4%). The compound is obtained as a racemic mixture of the expected (R) and(S) isomers.

### N-(4-((1,3-dioxoisoindolin-5-yl)oxy)phenyl)-3-methoxybenzamide (A68-6)

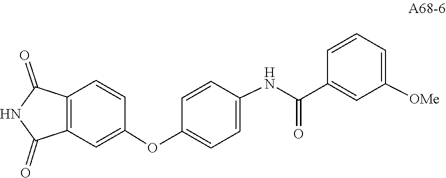 [SMILES: COc1cccc(C(=O)Nc2ccc(Oc3ccc4c(c3)C(=O)NC4=O)cc2)c1]

### N-(4-((1,3-dioxoisoindolin-5-yl)oxy)phenyl)-2-methoxybenzamide (A68-8)

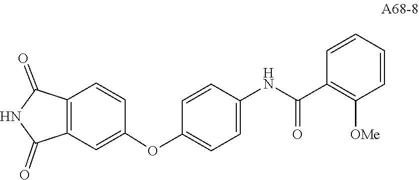

### 3-methoxy-N-(4-phenoxyphenyl)benzamide (A68-16)

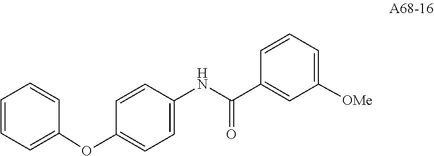 [SMILES: COc1cccc(C(=O)Nc2ccc(Oc3ccccc3)cc2)c1]

### 3-methoxy-N-(4-(4-methoxyphenoxy)phenyl)benzamide (A68-17)

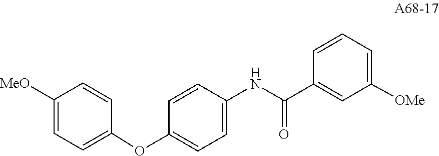 [SMILES: COc1ccc(Oc2ccc(NC(=O)c3cccc(OC)c3)cc2)cc1]

### 3-methoxy-N-(4-(2-methoxyphenoxy)phenyl)benzamide (A68-18)

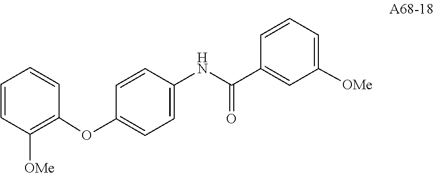 [SMILES: COc1cccc(C(=O)Nc2ccc(Oc3ccccc3OC)cc2)c1]

### N-(4-((1,3-dioxoisoindolin-5-yl)oxy)phenyl)thiophene-2-carboxamide (A68-24)

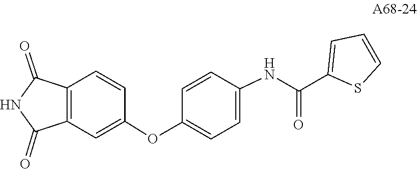 [SMILES: O=C(Nc1ccc(Oc2ccc3c(c2)C(=O)NC3=O)cc1)c1cccs1]

### N-(4-((1,3-dioxoisoindolin-5-yl)oxy)phenyl)-2-phenylacetamide (A68-37)

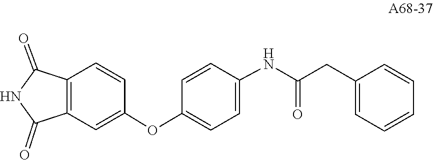 [SMILES: O=C(Cc1ccccc1)Nc1ccc(Oc2ccc3c(c2)C(=O)NC3=O)cc1]

### N-(4-((1,3-dioxoisoindolin-5-yl)oxy)phenyl)-2-(p-tolyloxy)acetamide (A68-38)

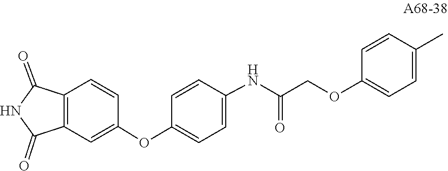 [SMILES: Cc1ccc(OCC(=O)Nc2ccc(Oc3ccc4c(c3)C(=O)NC4=O)cc2)cc1]

### 3-((4-((1,3-dioxoisoindolin-5-yl)oxy)phenyl) carbamoyl)phenyl acetate (A68-39)

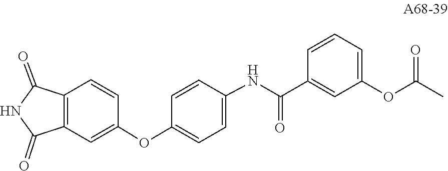 [SMILES: CC(=O)Oc1cccc(C(=O)Nc2ccc(Oc3ccc4c(c3)C(=O)NC4=O)cc2)c1]

### N-(4-((1,3-dioxo-2-((tetrahydrofuran-2-yl)methyl) isoindolin-5-yl)oxy)phenyl)-3-methoxybenzamide (A68-53)

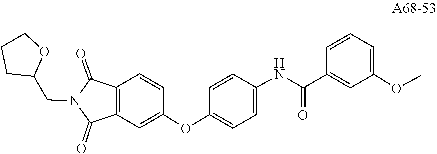 [SMILES: COc1cccc(C(=O)Nc2ccc(Oc3ccc4c(c3)C(=O)N(CC3CCCO3)C4=O)cc2)c1]

Note: this compound is racemic, 2 stereoisomers exit:

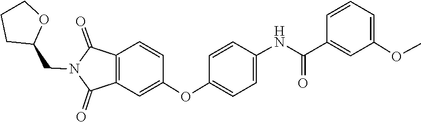 [SMILES: COc1cccc(C(=O)Nc2ccc(Oc3ccc4c(c3)C(=O)N(C[C@H]3CCCO3)C4=O)cc2)c1]

### (R)—N-(4-((1,3-dioxo-2-((tetrahydrofuran-2-yl)methyl) isoindolin-5-yl)oxy)phenyl)-3-methoxybenzamide

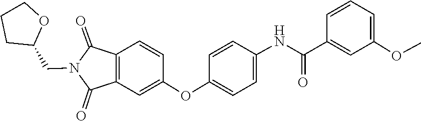 [SMILES: COc1cccc(C(=O)Nc2ccc(Oc3ccc4c(c3)C(=O)N(C[C@@H]3CCCO3)C4=O)cc2)c1]

### (S)—N-(4-((1,3-dioxo-2-((tetrahydrofuran-2-yl)methyl) isoindolin-5-yl)oxy)phenyl)-3-methoxybenzamide

### N-(4-((1,3-dioxoisoindolin-5-yl)oxy)phenyl)-2-(phenylthio)acetamide (A68-55)

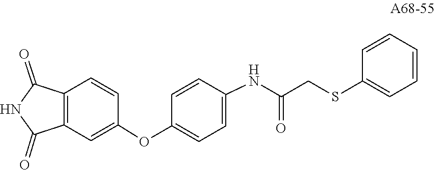 [SMILES: O=C(CSc1ccccc1)Nc1ccc(Oc2ccc3c(c2)C(=O)NC3=O)cc1]

### N-(4-((1,3-dioxoisoindolin-5-yl)oxy)phenyl)-3-methylbenzamide (A68)

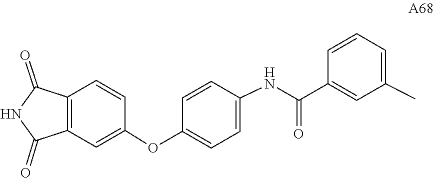 [SMILES: Cc1cccc(C(=O)Nc2ccc(Oc3ccc4c(c3)C(=O)NC4=O)cc2)c1]

1. A compound depicted in formula (I) 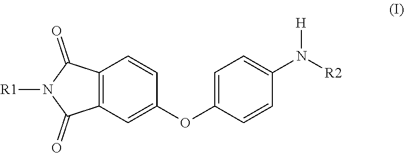
wherein R1 is H or 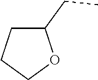
and wherein R2 is selected from the list consisting of 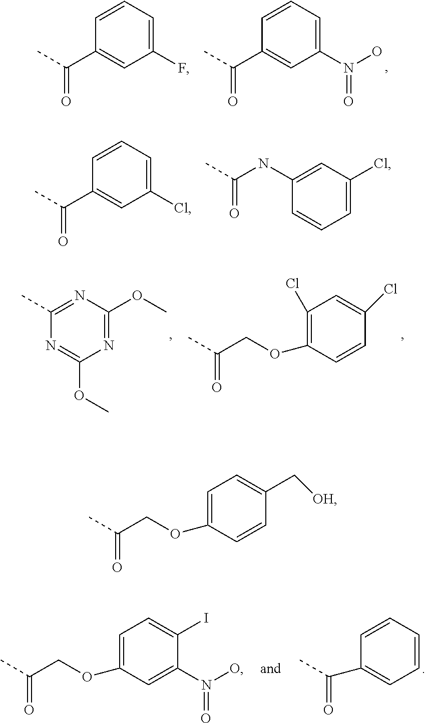
wherein the bound depicted as [symbol] connects the structure with the nitrogen (N—H group), and agronomically acceptable salts thereof, and individual enantiomers and diastereomers thereof.

2. A method of increasing the abiotic stress tolerance of a plant, the method comprising administering to the plant a compound depicted in formula (II) as 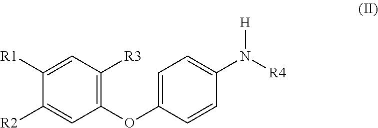
wherein R1 is H or 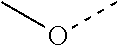
and R2 is H,
or wherein R1 and R2 together form an N-substituted maleimide structure: 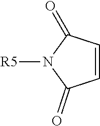
wherein R5 is H or 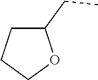
wherein the bound depicted as [symbol] connects the structure with the nitrogen (N—)
and wherein R3 is H or 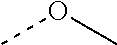
wherein the bound depicted as [symbol] connects the structure with the aryl moiety
and wherein R4 is 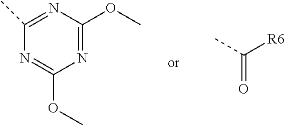
and wherein R6 is selected from the list consisting of 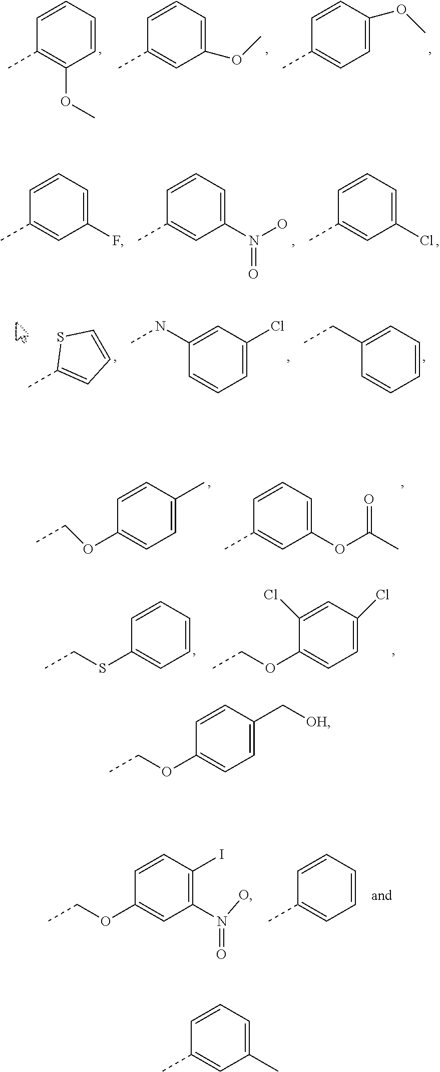
wherein the bound depicted as [symbol] connects the structures with the amide moiety, and the use of agronomically acceptable salts of these structures, and individual enantiomers and diastereomers thereof, for increasing the abiotic stress tolerance in plants.

3. The compound of claim 1, wherein the compound is selected from the list N-(4-((1,3-dioxoisoindolin-5-yl)oxy)phenyl)-3-fluorobenzamide, N-(4-((1,3-dioxoisoindolin-5-yl)oxy)phenyl)-3-nitrobenzamide, 3-chloro-N-(4-((1,3-dioxoisoindolin-5-yl)oxy)phenyl)benzamide, 1-(3-chlorophenyl)-3-(4-((1,3-dioxoisoindolin-5-yl)oxy)phenyl) urea, 5-(4-((4,6-dimethoxy-1,3,5-triazin-2-yl)amino) phenoxy) isoindoline-1,3-dione, 2-(2,4-dichlorophenoxy)-N-(4-((1,3-dioxoisoindolin-5-yl)oxy)phenyl)acetamide, N-(4-((1,3-dioxoisoindolin-5-yl)oxy)phenyl)-2-(4-(hydroxymethyl) phenoxy)acetamide, N-(4-((1,3-dioxoisoindolin-5-yl)oxy)phenyl)-2-(3-iodo-4-nitrophenoxy)acetamide, N-(4-((1,3-dioxoisoindolin-5-yl)oxy)phenyl)benzamide, and N-(4-((1,3-dioxo-2-((tetrahydrofuran-2-yl)methyl) isoindolin-5-yl)oxy)phenyl)benzamide.

4. The method according to claim 2, wherein the compound is selected from the list N-(4-((1,3-dioxoisoindolin-5-yl)oxy)phenyl)-3-fluorobenzamide, N-(4-((1,3-dioxoisoindolin-5-yl)oxy)phenyl)-3-nitrobenzamide, 3-chloro-N-(4-((1,3-dioxoisoindolin-5-yl)oxy)phenyl)benzamide, 1-(3-chlorophenyl)-3-(4-((1,3-dioxoisoindolin-5-yl)oxy)phenyl) urea, 5-(4-((4,6-dimethoxy-1,3,5-triazin-2-yl)amino) phenoxy) isoindoline-1,3-dione, 2-(2,4-dichlorophenoxy)-N-(4-((1,3-dioxoisoindolin-5-yl)oxy)phenyl)acetamide, N-(4-((1,3-dioxoisoindolin-5-yl)oxy)phenyl)-2-(4-(hydroxymethyl) phenoxy)acetamide, N-(4-((1,3-dioxoisoindolin-5-yl)oxy)phenyl)-2-(3-iodo-4-nitrophenoxy)acetamide, N-(4-((1,3-dioxoisoindolin-5-yl)oxy)phenyl)benzamide, N-(4-((1,3-dioxo-2-((tetrahydrofuran-2-yl)methyl) isoindolin-5-yl)oxy)phenyl)benzamide, N-(4-((1,3-dioxoisoindolin-5-yl)oxy)phenyl)-3-methoxybenzamide, N-(4-((1,3-dioxoisoindolin-5-yl)oxy)phenyl)-2-methoxybenzamide, 3-methoxy-N-(4-phenoxyphenyl)benzamide, 3-methoxy-N-(4-(4-methoxyphenoxy)phenyl)benzamide, and 3-methoxy-N-(4-(2-methoxyphenoxy)phenyl)benzamide, N-(4-((1,3-dioxoisoindolin-5-yl)oxy)phenyl)thiophene-2-carboxamide, N-(4-((1,3-dioxoisoindolin-5-yl)oxy)phenyl)-2-phenylacetamide, N-(4-((1,3-dioxoisoindolin-5-yl)oxy)phenyl)-2-(p-tolyloxy)acetamide, 3-((4-((1,3-dioxoisoindolin-5-yl)oxy)phenyl) carbamoyl)phenyl acetate, N-(4-((1,3-dioxo-2-((tetrahydrofuran-2-yl)methyl) isoindolin-5-yl)oxy)phenyl)-3-methoxybenzamide, N-(4-((1,3-dioxoisoindolin-5-yl)oxy)phenyl)-2-(phenylthio)acetamide, and N-(4-((1,3-dioxoisoindolin-5-yl)oxy)phenyl)-3-methylbenzamide.

5. A method for increasing the cold tolerance or frost tolerance of a plant, the method comprising administering to the plant a compound depicted in formula (II) as 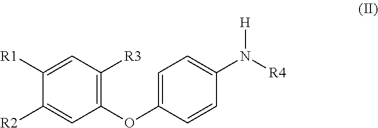
wherein R1 is H or 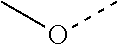
and R2 is H,
or wherein R1 and R2 together form an N-substituted maleimide structure: 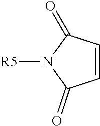
wherein R5 is H or 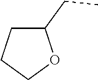
wherein the bound depicted as [symbol] connects the structure with the nitrogen (N—)
and wherein R3 is H or 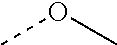
wherein the bound depicted as [symbol] connects the structure with the aryl moiety
and wherein R4 is 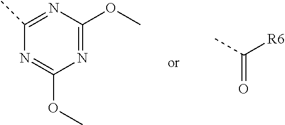
and wherein R6 is selected from the list consisting of 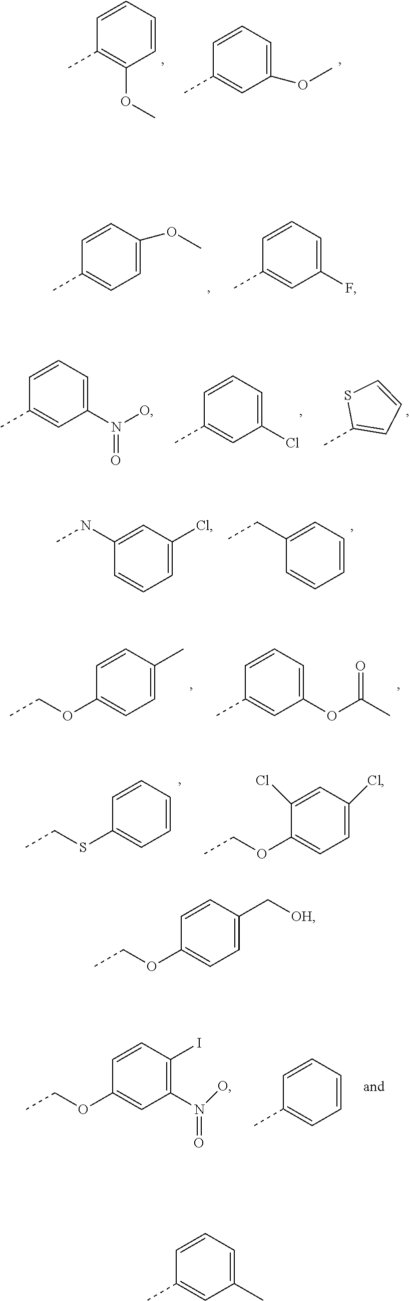
wherein the bound depicted as [symbol] connects the structures with the amide moiety, and the use of agronomically acceptable salts of these structures, and individual enantiomers and diastereomers thereof.

6. (canceled)

7. The method according to claim 5, wherein the compound is selected from the group consisting of N-(4-((1,3-dioxoisoindolin-5-yl)oxy)phenyl)-3-fluorobenzamide, N-(4-((1,3-dioxoisoindolin-5-yl)oxy)phenyl)-3-nitrobenzamide, 3-chloro-N-(4-((1,3-dioxoisoindolin-5-yl)oxy)phenyl)benzamide, 1-(3-chlorophenyl)-3-(4-((1,3-dioxoisoindolin-5-yl)oxy)phenyl) urea, 5-(4-((4,6-dimethoxy-1,3,5-triazin-2-yl)amino) phenoxy) isoindoline-1,3-dione, 2-(2,4-dichlorophenoxy)-N-(4-((1,3-dioxoisoindolin-5-yl)oxy)phenyl)acetamide, N-(4-((1,3-dioxoisoindolin-5-yl)oxy)phenyl)-2-(4-(hydroxymethyl) phenoxy)acetamide, N-(4-((1,3-dioxoisoindolin-5-yl)oxy)phenyl)-2-(3-iodo-4-nitrophenoxy)acetamide, N-(4-((1,3-N-(4-((1,3-dioxo-2-((tetrahydrofuran-2-dioxoisoindolin-5-yl)oxy)phenyl)benzamide, yl)methyl) isoindolin-5-yl)oxy)phenyl)benzamide, N-(4-((1,3-dioxoisoindolin-5-yl)oxy)phenyl)-3-methoxybenzamide, N-(4-((1,3-dioxoisoindolin-5-yl)oxy)phenyl)-2-methoxybenzamide, 3-methoxy-N-(4-phenoxyphenyl)benzamide, 3-methoxy-N-(4-(4-methoxyphenoxy)phenyl)benzamide, and 3-methoxy-N-(4-(2-methoxyphenoxy)phenyl)benzamide, N-(4-((1,3-dioxoisoindolin-5-yl)oxy)phenyl)thiophene-2-carboxamide, N-(4-((1,3-dioxoisoindolin-5-yl)oxy)phenyl)-2-phenylacetamide, N-(4-((1,3-dioxoisoindolin-5-yl)oxy)phenyl)-2-(p-tolyloxy)acetamide, 3-((4-((1,3-dioxoisoindolin-5-yl)oxy)phenyl) carbamoyl)phenyl acetate, N-(4-((1,3-dioxo-2-((tetrahydrofuran-2-yl)methyl) isoindolin-5-yl)oxy)phenyl)-3-methoxybenzamide, N-(4-((1,3-dioxoisoindolin-5-yl)oxy)phenyl)-2-(phenylthio)acetamide, and N-(4-((1,3-dioxoisoindolin-5-yl)oxy)phenyl)-3-methylbenzamide.
